(19) United States
(12) Patent Application Publication
(10) Pub. No.: US 2016/0206245 A1
(43) Pub. Date: Jul. 21, 2016

(54) ASYMMETRIC GAIN OF CAPACITIVE SENSORS FOR MEASURING PHYSIOLOGICAL PARAMETERS

(71) Applicant: Covidien LP, Mansfield, MA (US)

(72) Inventors: Eric Morland, Erie, CO (US); Christopher J. Meehan, Arvada, CO (US); Philip Davis, El Paso, TX (US); Tim Fries, Louisville, CO (US); Daniel Lisogurski, Boulder, CO (US); Clark R. Baker, JR., Newman, CA (US); Rasoul Yousefi, Superior, CO (US)

(21) Appl. No.: 14/996,438

(22) Filed: Jan. 15, 2016

Related U.S. Application Data
(60) Provisional application No. 62/105,899, filed on Jan. 21, 2015.

(51) Int. Cl. A61B 5/00; A61B 5/1455; A61B 5/053

(52) U.S. Cl. CPC A61B 5/721; A61B 5/053; A61B 5/14552; A61B 2562/0214

(57) ABSTRACT

Systems, methods, sensors, and software for providing enhanced measurement and correction of physiological data are provided herein. In one example, a capacitive sensor of a measurement system is positioned onto tissue of a patient. The capacitive sensor includes one or more conductive elements with associated gain properties that are positioned near optical sensor elements proximate to the tissue of the patient, the optical sensor elements positioned to measure a photoplethysmogram (PPG) for the tissue. The measurement system drives the capacitive sensor and measures capacitance signals associated with the capacitance sensor. The measurement system corrects for at least motion noise in the PPG using the capacitance signals.

### RELATED APPLICATIONS

This application hereby claims the benefit of priority to U.S. Provisional Patent Application 62/105,899, titled “ASYMMETRIC GAIN OF CAPACITIVE SENSORS FOR MEASURING PHYSIOLOGICAL PARAMETERS,” filed Jan. 21, 2015, which is hereby incorporated by reference in its entirety.

### TECHNICAL FIELD

Aspects of the disclosure are related to the field of medical devices, and in particular, measuring physiological parameters and correcting measured physiological parameters, such as plethysmograms.

### BACKGROUND

Various medical devices can non-invasively measure parameters of blood in a patient. Pulse oximetry devices are one such non-invasive measurement device, typically employing solid-state lighting elements, such as light-emitting diodes (LEDs) or LED lasers, to introduce light into the tissue of a patient. The light is then detected to generate a photoplethysmogram (PPG). These photoplethysmography systems can also measure changes in blood volume of tissue of a patient and calculate various parameters such as heart rate, respiration rate, and oxygen saturation.

However, conventional optical pulse oximetry devices are subject to motion noise and other inconsistencies which limit the accuracy of such devices. For example, motion of the patient and movement of nearby objects or medical personnel can lead to noise and inaccuracies of optical-based measurements. This noise in the photoplethysmogram data can lead to false pulse reporting, inaccurate physiological data, or prevent measurement of the patient until motion noise subsides.

Capacitive sensing has been employed to measure some physiological parameters by applying electric fields to the tissue of the patient. However, these capacitive systems rely upon conventional capacitor plate configurations, such as flat, solid plates, and still suffer from noise and inconsistencies due to not only motion of the patient, but also motion of nearby objects and personnel.

### OVERVIEW

Systems, methods, sensors, and software for providing enhanced measurement and correction of physiological data are provided herein. In a first example, a capacitive sensor of a measurement system is positioned onto tissue of a patient. The capacitive sensor includes one or more conductive elements with associated asymmetric gain properties that are positioned near optical sensor elements proximate to the tissue of the patient, the optical sensor elements positioned to measure a photoplethysmogram (PPG) for the tissue. The measurement system drives the capacitive sensor and measures capacitance signals associated with the capacitance sensor. The measurement system corrects for at least motion noise in the PPG using the capacitance signals.

In a second example, a physiological sensor configured to be positioned onto tissue of a patient is provided. The sensor includes a first conductive element with an associated first gain property and disposed about at least an optical emitter. The sensor includes a second conductive element with an associated second gain property and disposed about at least an optical detector. The sensor includes a sensor body coupled to at least the first conductive element and the second conductive element and configured to interface with tissue of the patient.

In a third example, a measurement system employing an asymmetric capacitive sensor system to reduce noise in a photoplethysmogram (PPG) derived from an optical signal propagated through tissue of a patient is provided. The measurement system includes a capacitance system configured to measure a first capacitance signal from a first capacitance element positioned proximate to an optical emitter that emits the optical signal into the tissue of the patient. The capacitance system is configured to measure a second capacitance signal from a second capacitance element positioned proximate to an optical detector that detects at least the optical signal after propagation through the tissue of the patient, the second capacitance element having an associated gain asymmetric to that of the first capacitance element. The measurement system includes a processing system configured to compare the first capacitance signal to the second capacitance signal to identify a differential capacitance signal. The processing system is configured to identify a corrected PPG by at least reducing a magnitude of noise components in the PPG based at least in part on the differential capacitance signal.

In a fourth example, a physiological measurement apparatus is provided. The apparatus includes a generally ring-shaped first capacitor plate configured to interface with tissue of a patient to emit an electric field proximate to the tissue of the patient, the first capacitor plate having a first associated gain property. The apparatus includes a generally ring-shaped second capacitor plate configured to interface with the tissue of the patient and having a second associated gain property different than the first gain property. The apparatus includes a measurement system electrically coupled to the first capacitor plate and the second capacitor plate and configured to generate an electric signal referenced to a ground potential, drive the electric signal to the first capacitor plate for emission as the electric field, electrically couple the second capacitor plate to the ground potential, monitor properties of the electric signal during emission into the tissue of the patient to identify a capacitance signal associated with the first capacitor plate, and process the capacitance signal to determine one or more physiological metrics associated with the patient.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 is a system diagram illustrating a physiological measurement system.

FIGS. 2A and 2B are flow diagrams illustrating methods of operating a physiological measurement system.

FIG. 3 is a system diagram illustrating a physiological measurement system.

FIG. 4 illustrates various measured and corrected signals for a patient.

FIG. 5 is a system diagram illustrating a physiological sensor.

FIG. 6 is a block diagram illustrating a physiological measurement system.

### DETAILED DESCRIPTION

The examples discussed herein include systems, apparatuses, methods, and software for enhanced measurement of physiological parameters in patients. When certain measurements of patient data are performed, such as optical measurements, signals associated with the measurements can be subjected to various interference and noise due to patient motion, among other sources of noise. For example, motion noise occurs in pulse oximetry measurements due in part to optical emitter-detector spacing changes, light coupling changes, deformation of the tissue under measurement, and changes in venous blood volume, among other motion noise sources. It can be difficult to reduce the noise caused by motion during optical measurements. However, capacitance-based sensing can be employed in conjunction with optical measurements to provide for effective filtering and noise correction of the optical signals. This capacitance-based sensing can be employed to enhance or supplement these measurements to provide correction, filtering, data stabilization, or additional sensing capabilities to other measurement systems.

The examples discussed herein employ one or more generally ring-shaped capacitor plates. These ring-shaped capacitor plates maximize sensitivity to motion noise, but minimize sensitivity to changes in venous blood volume of the tissue, such as due to pulse. Signals measured by the ring-shaped capacitor plates include motion noise which can be used to cancel out or reduce similar motion noise in optical signals. The ring-shaped capacitor plates allow pulsatile changes in the tissue under measurement to minimally affect the capacitor plates, while still allowing for detection of bulk movements of the tissue, such as due to pressing, squeezing, flexing, and clenching of the tissue. Also, in many of the examples below, an asymmetric gain is applied to the ring-shaped capacitor plates, which can enhance signal detection when motion noise occurs equally on two ring-shaped capacitor plates which might be otherwise be canceled out in a differential detection mode.

Although many of the examples herein discuss ring-shaped capacitor plates, it should be understood that different shapes can be employed. For example, any polygonal shape can be employed, which may include filled conductive areas or banded perimeters of conductive material, including combinations thereof. Also, although a differential signal processing technique is employed in many examples, it should be understood that other mathematical operations or combinations of one or more measurement signals can be used, such as subtraction, addition, multiplication, division, exponential, composite polynomial functions, complex algebraic combinations, or other mathematical operations, including combinations thereof.

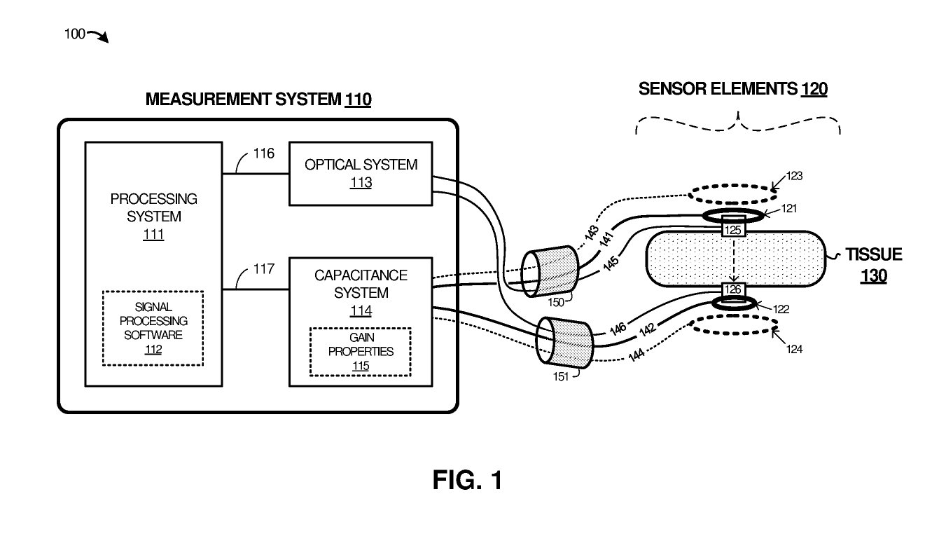

As a first example of a measurement system for monitoring physiological parameters of a patient, FIG. 1 is presented. FIG. 1 is a system diagram illustrating physiological system 100. Elements of physiological system 100 measure one or more physiological parameters of tissue 130. In the example shown in FIG. 1, physiological system 100 includes measurement system 110, sensor elements 120, and tissue 130. In operation, sensor elements 120 are configured to monitor various properties of tissue 130 and provide signals indicating these properties to measurement system 110 for processing and analysis.

Measurement system 110 includes processing system 111, optical system 113, and capacitance system 114. Processing system 111 and optical system 113 communicate over link 116. Processing system 111 and capacitance system 114 communicate over link 117. Links 116 and 117 can each comprise one or more analog or digital links. Measurement system 110 includes both optical measurement equipment and capacitive measurement equipment, as represented in FIG. 1 by optical system 113 and capacitance system 114, respectively.

Turning first to the capacitive sensing elements of FIG. 1, capacitance system 114 monitors physiological signals associated with tissue 130 using capacitive sensing elements 121 or 122. For example, capacitance system 114 can drive electrical signals over links 141 and 142 and detect changes in those electrical signals to monitor tissue 130. Capacitance system 114 can drive an oscillating or alternating current (AC) signal onto link 141 for emission by capacitive element 121 proximate to tissue 130. Likewise, capacitance system 114 can drive an oscillating AC signal onto link 142 for emission by capacitive element 122 proximate to tissue 130. Capacitance system 114 detects changes in signals driven onto link 141 and 142, such as current draws which correspond to capacitance value changes. Differential and non-differential measurement schemes may be employed, and one or more of capacitive elements 121 and 122 can be electrically grounded to establish a voltage potential between capacitive elements and tissue 130. A single capacitive element might be employed instead of the dual capacitive configuration shown in FIG. 1.

Furthermore, an asymmetric capacitance sensing configuration is employed in conjunction with optical sensing of tissue 130. The asymmetric capacitance configuration includes one or more capacitance elements with different associated gain properties. The gain properties can be established based on geometric properties of capacitive sensing elements of sensor elements 120, such as a size of the conductive area, as discussed below, or based on gain properties applied in hardware amplification elements 115 or software processing elements 112, including combinations thereof.

The asymmetric gain can help compensate for measurements when physical properties of the tissue under measurement are asymmetric. For example, when a finger is the tissue under measurement, different properties of different sides of the finger might factor into the asymmetric gain, such as when one side of the finger has different moisture or elasticity properties than the other side. Other sites of a patient body may have more symmetrical physical properties when using two or more capacitive plates, such as in a side-by-side configuration on a forehead or chest, or on fingers of infants with more symmetrical physical properties due to reduced fingernail development. Selection of an asymmetric or symmetric measurement technique can vary based on the location on the patient that measurements are performed, or upon which technique leads to more effective measurement of the desired signals.

As mentioned above, one or more capacitive sensing elements are employed in system 100 in cooperation with measurement system 110. The capacitive sensing elements include generally circular or ring-shaped capacitive elements 121 and 122. The ring-shaped configuration of capacitive elements 121 and 122 are shown as having a different conductive area in FIG. 1. These different conductive areas, based in part on the diameters of capacitive elements 121 and 122, establish a different gain for capacitive elements 121 and 122. The different diameters can lead to different gains by having differing quantities of conductive material in proximity to tissue 130 which can lead to different capacitances associated with the different diameters. In examples where ring-shaped capacitive elements are employed, thicknesses of the ring-shaped conductive bands that form the ring-shaped capacitive elements can also be selected to establish a desired conductive area, such as by selecting inner diameter and outer diameter dimensions. In alternate configurations, a similar or the same conductive area can be employed for capacitive elements 121 and 122 and a gain correction or gain differential can be applied by elements of measurement system 110. Also, in further examples, the generally circular ring shapes of capacitive elements 121 and 122 might vary and instead be rectangular-shaped rings, triangular rings, or oval or elliptical rings, among other shapes and sizes. Although the particular physical arrangement of capacitive elements 121 and 122 are shown as on opposite sides of tissue 130 in FIG. 1, it should be understood that various arrangements can be employed, such as on the same side of tissue 130.

One or more shield elements 123 and 124 can be employed to electrically shield an associated capacitive element from external electric fields or to directionally attenuate electric fields emitted by the associated capacitive element. Shield elements 123 and 124 can be linked to capacitance system via associated links 143 and 144. Links 143 and 144 can provide for electrical grounding of shields 123 and 124, or to allow for electrical signals to be driven onto shields 123 and 124 by elements of capacitance system 114. Also, one or more cable shields 150 and 151 can be employed over ones of links 141-145 to electrically shield the links from external electric fields and attenuate electric emissions of the links, among other functions such as structural support and physical protection. Cable shields 150 and 151 can also be coupled to electrical ground potentials at one or both ends, or actively driven with electrical signals by capacitance system 114.

Continuing with the discussion of the elements of FIG. 1, optical system 113 measures various properties of tissue 130 using optical emitter 125 and optical detector 126. In some examples, optical system 113, along with optical emitter 125 and optical detector 126, comprise a pulse oximeter and can identify a photoplethysmogram (PPG) for tissue 130. This PPG can be used to determine various properties of tissue 130 or the patient associated with tissue 130, such as changes in blood volume of tissue 130 which correspond to various parameters such as pulse rate, respiration rate, and oxygen saturation, among other parameters.

Optical system 113 drives signals over link 145 to optical emitter 125. Optical emitter 125 emits optical signals into tissue 130 for propagation through tissue 130. Optical detector 126 detects these optical signals after propagation through tissue 130. Optical system 113 receives signals over link 146 from optical detector 126. The signals on links 145 and 146 can comprise optical signals when links 145 and 146 comprise optical fiber links, or can comprise electrical signals when links 145 and 146 comprise electrical links. Various combinations of optical and electrical signaling can be employed between any of optical emitter 125 and optical detector 126 and optical system 113.

In some examples, link 145 is a wired or wireless signal link, and carries a measurement signal to optical emitter 125, and optical emitter 125 converts the measurement signal into an optical signal and emits an optical signal into tissue 130. The optical signal can be emitted using a laser, laser diode, light emitting diode (LED), or other light emission device. In other examples, link 145 is an optical link, and carries an optical signal to optical emitter 125. Optical emitter 125 can comprise tissue interface optics, such as lenses, prisms, or other optical fiber-to-tissue optics, which interface to tissue 130 for emission of optical signals. One or more optical wavelengths can be introduced by optical emitter 125 into tissue 130, and the one or more optical wavelengths can be selected based on various physiological factors, such as isosbestic wavelengths associated with blood components of tissue 130. In a particular example, wavelengths such as 660 nm and 808 nm are employed.

Optical detector 126 detects the optical signals after propagation through tissue 130. Optical system 113 receives signals over link 146 from optical detector 126 representative of the optical signal after propagation through tissue 130. In some examples, link 146 is a wired or wireless signal link, and carries a signal from optical detector 126, where optical detector 126 converts detected optical signals into associated electrical signals. Detector 126 can comprise a photodiode, avalanche photodiode, or other optical detection device. In other examples, link 146 is an optical link, and carries an optical signal from optical detector 126. Optical detector 126 can comprise tissue interface optics, such as described above for optical emitter 125, which interface to tissue 130.

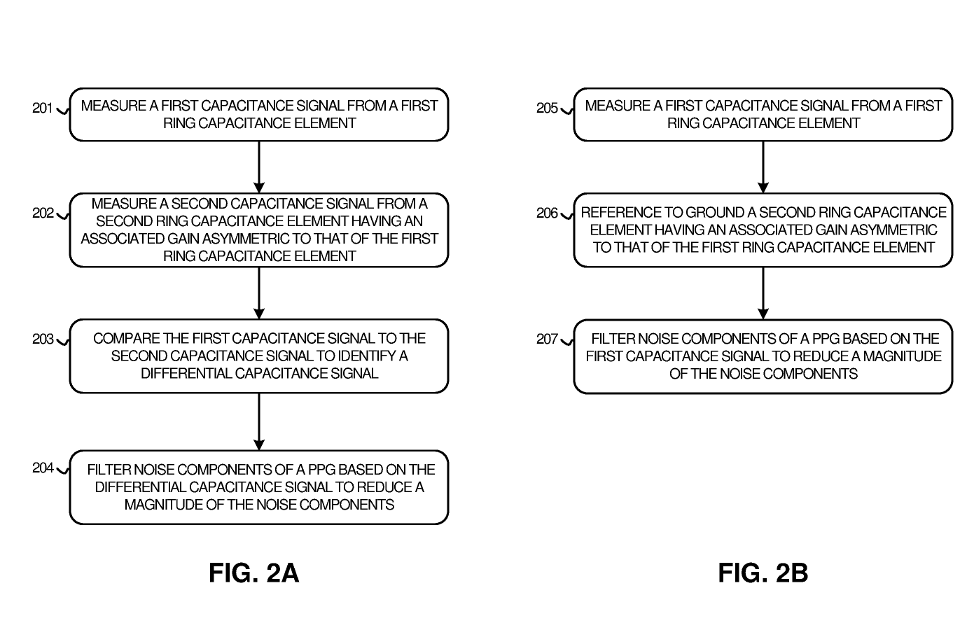

To illustrate the operation of the elements of FIG. 1, FIGS. 2A and 2B are provided. FIG. 2A is a flow diagram illustrating a method of operating measurement system 110 using a differential capacitance arrangement. FIG. 2B is a flow diagram illustrating a method of operating measurement system 110 using a non-differential capacitance arrangement. The operations of FIGS. 2A and 2B are referenced below parenthetically. Although FIG. 1 shows ring-shaped capacitor plates, it should be understood that different shapes can be employed. For example, any polygonal shape can be employed, which may include filled conductive areas or banded perimeters of conductive material, including combinations thereof.

Turning first to FIG. 2A, measurement system 110 measures (201) a first capacitance signal from a first ring capacitance element. Specifically, in FIG. 1, measurement system 110 measures the first capacitance signal using capacitance element 121. Capacitive element 121 is disposed about optical emitter 125. A capacitance measurement is performed generally concurrent with an optical measurement of tissue 130 by measurement system 110. The capacitive measurement includes, in this example, capacitance system 114 driving an AC signal onto link 141 which is emitted by capacitance element 121 as an electric field proximate to capacitance element 121. Capacitance system 114 can detect variations in a current draw of the AC signal over time which can correspond to a varying capacitance of capacitive element 121. This time-varying capacitance signal for capacitance element 121 is then employed by processing system 111 as discussed below.

Although FIG. 1 shows capacitive element 121 disposed about optical emitter 125, other configurations are possible. For example, capacitive element 121 (or alternatively capacitive element 122) can be disposed about both optical emitter 125 and optical detector 126. Alternatively, capacitive element 121 and capacitive element 122 might not be disposed about any of optical emitter 125 and optical detector 126.

Measurement system 110 measures (202) a second capacitance signal from a second ring capacitance element having an associated gain asymmetric to that of the first ring capacitance element. Specifically, in FIG. 1, measurement system 110 measures the second capacitance signal from capacitance element 122. Capacitive element 122 is disposed about optical detector 126. A second capacitance measurement is performed generally concurrent with the first capacitive measurement discussed in operation 201 and the optical measurement. The second capacitive measurement includes, in this example, capacitance system 114 driving an AC signal onto link 142 which is emitted by capacitance element 122 as an electric field proximate to capacitance element 122. Capacitance system 114 can detect variations in a current draw of the AC signal over time which can correspond to a varying capacitance of capacitive element 122. This time-varying capacitance signal for capacitance element 122 is then employed by processing system 111 as discussed below. Other detection schemes for measuring a varying capacitance of capacitive elements 121 and 122 can be performed instead of using the current draw example mentioned herein.

In this example, capacitance element 122 has a different gain associated therewith which is asymmetric than the gain of capacitance element 121. As discussed above, this different gain can be established by providing a different conductive area of each of capacitance elements 121 and 122, such as by having a different diameter. Alternatively, or in combination, a hardware gain can be employed to signals associated with each of capacitance elements 121 and 122 during conditioning, filtering, or amplification, analog-to-digital conversion in capacitance system 114. Furthermore, a software gain can be employed to data associated with each of capacitance elements 121 and 122 during processing and analysis by processing system 111, such as in signal processing software 112.

The asymmetric gains for capacitance elements 121 and 122 can be established to maximize resolution of bulk motion noise associated with tissue 130 while minimizing resolution of motion noise due to the pulse within tissue 130. The gains can be selected based on desired frequency sensitivity of these various types of motion noise, such as to minimize sensitivity to frequency ranges associated with pulse motion within tissue 130, while maximizing sensitivity to frequency ranges associated with bulk movement of tissue 130 within the environment. For example, bulk movement can be found to occur within a first range of frequencies while pulse motion can be found to occur in a second range of frequencies. In one example, bulk motion might occur around a frequency of 4 Hz, while pulse motion might occur around a frequency of 1 Hz. The gains can be selected for sensitivity to either the bulk motion or the pulse motion, depending upon which motion is presently being characterized. Empirical measurements of capacitive signals can be performed on the patient or prior to measurement of tissue 130 which can establish desired gains or calibrate the gains applied to capacitance signals on a per-patient basis, such as to maximize sensitivity of the capacitance signals to bulk motion of a specific patient and minimize sensitivity of the capacitance signals to pulse motion of that patient. The empirical measurements can be made to steer adjustments to electrical/software gains or physical sizing of capacitance elements to optimize signal characteristics associated with tissue of the patient. Similarly, a sizing of capacitance elements 121 and 122 can be established based on a desired gain, differential gain, or upon sensitivity to certain frequency components associated with motion noise.

In one example, a conductive area of capacitance element 122 is less than a conductive area of capacitance elements 121, such as capacitance element 122 having a diameter of 1.5 centimeters (cm) and capacitance element 121 having a diameter of 1.0 cm. It should be understood that other diameters can be selected, including the same diameter if the different gains are applied in downstream hardware or software. Additionally, a reverse filtering method can be employed to select conductive areas or gains of capacitance elements 121 and 122 that maximize or enhance motion noise components of desired frequency or temporal characteristics. For example, a pulse signal can be filtered out of optical and capacitance measurements and a bulk motion signal can be monitored to identify gains or conductive areas that correspond to maximum energy of the bulk motion signal. In yet further examples, an array of selectable capacitive elements can be employed, where ones of the array are selected as needed based on gain preferences for the tissue under measurement. The array of selectable capacitive elements can be selected using electrical switching techniques to select different elements or to select an amount of area or size of diameter of capacitive elements to use in measurement.

Measurement system 110 compares (203) the first capacitance signal to the second capacitance signal to identify a differential capacitance signal. As discussed above, the first capacitance signal is measured using capacitance element 121 and the second capacitance signal is measured using capacitance element 122. An asymmetric gain is applied to these capacitance signals, whether using geometry, hardware gain, or software gain, and a differential capacitance signal is determined. The differential capacitance signal can be determined by processing system 111 or by capacitive system 114 using various signal processing techniques. The differential capacitance signal might be a subtraction of one of the capacitance signals from the other capacitance signal. The subtraction can be performed using hardware elements in capacitance system 114, or using signal processing software 112 of processing system 111 to process data derived from digitization of the two capacitance signals.

Measurement system 110 filters (204) noise components of a photoplethysmogram (PPG) based on the differential capacitance signal to reduce a magnitude of the noise components of the PPG. As mentioned above, an optical measurement of tissue 130 is performed generally concurrent with capacitance measurements of operations 201 and 202. These optical measurements produce a PPG which indicates optically-measured signals of tissue 130. However, the PPG can include various noise components due to various sources of noise, such as noise caused by motion of the patient associated with tissue 130. These noise components might prevent determination of physiological parameters from the PPG, such as pulse rate, breathing rate, or other parameters of the patient. The time-varying differential capacitance signal is employed to reduce the magnitude of these noise components in the PPG. In some examples with appropriately chosen differential gains, the differential capacitance signal can be processed to represent a time-varying bulk motion signal, capturing the frequencies of bulk movement unrelated to the desired pulsatile signal component. By using the differential capacitance signal to filter the PPG, motion noise found into the PPG can be reduced to allow a filtered or clean PPG to be further processed or displayed.

The filtering of the noise components of the PPG can include various types of filtering. In a first example, a frequency domain analysis is performed to identify frequency components in the differential capacitance signal that are related to motion noise of the patient. These frequency components can be filtered out of the PPG using bandpass filtering at the various frequencies associated with the motion noise. These frequency components can also be filtered out by subtracting the differential capacitance signal from the PPG, either in a frequency domain or in a time domain. Other techniques to filter the noise components of the PPG are discussed in the examples below.

Turning now do FIG. 2B, a non-differential measurement scheme will be discussed. The non-differential measurement scheme can include a single-ended measurement scheme using first capacitor plate referenced to a potential voltage, such as a ground potential voltage provided by a second capacitor plate. In FIG. 2B, measurement system 110 measures (205) a first capacitance signal from a first ring capacitance element. As with the examples in FIG. 2A, measurement system 110 measures the first capacitance signal using capacitance element 121. Capacitive element 121 is disposed about optical emitter 125. A capacitance measurement is performed generally concurrent with an optical measurement of tissue 130 by measurement system 110. The capacitive measurement includes, in this example, capacitance system 114 driving an AC signal onto link 141 which is emitted by capacitance element 121 as an electric field proximate to capacitance element 121. Capacitance system 114 can detect variations in a current draw of the AC signal over time which can correspond to a varying capacitance of capacitive element 121. This time-varying capacitance signal for capacitance element 121 is then employed by processing system 111 as discussed below.

Measurement system 110 references (206) to ground a second ring capacitance element having an associated gain asymmetric to that of the first ring capacitance element. In this examples, the second ring capacitance element is capacitance element 122 which is referenced to a ground potential by capacitance system 114. The ground potential is also common to the AC signal driving capacitance element 121. In this configuration, electrical fields emitted by capacitance element 121 are partially grounded by capacitance element 122. Measurement system 110 does not directly monitor a capacitance signal associated with capacitance element 122, and instead monitors the capacitance signal associated with capacitance element 121 which is affected by the ground potential introduced by capacitance element 122.

Capacitance element 122 has a different gain associated therewith which is asymmetric than the gain of capacitance element 121. However, in this example, the gain is established by a conductive area of each of capacitance elements 121 and 122, such as by having a different conductive area. For example, capacitance element 121 can be of a greater diameter than capacitive element 122. Conversely, capacitive element 121 could instead have a smaller diameter than capacitive element 122.

Measurement system 110 filters (207) noise components of a PPG based on the first capacitance signal to reduce a magnitude of the noise components. As mentioned above, an optical measurement of tissue 130 is performed concurrent with capacitance measurements of operation 205. These optical measurements produce a PPG which indicates optically-measured signals of tissue 130. However, the PPG can include various noise components due to various sources of noise, such as noise caused by motion of the patient associated with tissue 130. The time-varying single-ended capacitance signal is employed to reduce the magnitude of these noise components in the PPG. In this manner, motion noise introduced into the PPG can be reduced to allow a filtered or clean PPG to be further processed or displayed. A similar filtering process as in operation 204 can be employed, such as by signal subtraction or frequency domain analysis.

A differential capacitance signal, such as discussed in FIG. 2A, might lead to different signal components representing motion of the patient than a single-ended capacitance signal as discussed in FIG. 2B. An operator can select among the two measurement techniques to determine which is best for the particular measurement or the particular patient. For example, with many medical personnel in the vicinity of the patient, such as during a surgical procedure, one of the two techniques might lead to better motion cancelation than the other technique due to capacitive influence of nearby personnel or equipment. Likewise, when the patient is in a recovery setting with fewer medical personnel nearby, a different one of the two techniques might lead to better motion cancelation. Other factors can influence which of the two techniques discussed in FIGS. 2A and 2B are employed, such as the type or frequency of motion, nearby medical equipment interference, or patient-specific characteristics including tissue moisture content, bodily location of measurement, or other factors, including combinations thereof. Additionally, both techniques can be employed either simultaneously or in succession to further refine the measurement and motion cancelation, or to establish which technique is better suited for a particular application.

Once a filtered PPG is determined, such as in operations 204 or 207 of FIGS. 2A and 2B, various physiological parameters can be determined from the PPG alone or from the PPG in combination with the capacitance signals. The physiological parameters can include various plethysmograph (pleth) information, such as clean photoplethysmograms (PPG) and temporal variability of PPG parameters (such as pleth morphology and pulse information). The physiological parameters measured or determined by the capacitance-enhanced systems can also include electrocardiography (ECG) information via capacitive sensing, pulse rate, respiratory rate, respiratory effort, blood pressure, oxygen concentrations, hemoglobin concentrations, total hemoglobin concentration (tHb), saturation of peripheral oxygen (SpO₂), SpO₂ variability, regional oxygen saturation (rSO₂), apnea conditions, arrhythmia, and saturation pattern detection among other parameters and characteristics, including combinations and variations thereof. Physiological measurements can be performed using the various examples herein. Some of these include determining respiration rate from a finger, pulse rate from a finger, motion of patient, continuous non-invasive blood pressure measurement (CNIBP), deltaPOP (a measurement of the variability of the pleth pulses), variability of optical pleth to determine vessel elasticity, dehydration, apnea detection and monitoring, and auto-regulation of patients.

Returning to the elements of FIG. 1, either optical system 113 can include electrical to optical conversion circuitry and equipment, optical modulation equipment, and optical waveguide interface equipment. Optical system 113 can include direct digital synthesis (DDS) components, function generators, oscillators, or other signal generation components, filters, delay elements, signal conditioning components, such as passive signal conditioning devices, attenuators, filters, directional couplers, active signal conditioning devices, amplifiers, phase detectors, or frequency converters, including combinations thereof. Optical system 113 can also include switching, multiplexing, or buffering circuitry, such as solid-state switches, RF switches, diodes, or other solid state devices. Optical system 113 also can receive command and control information and instructions from processing system 111 over link 116 for controlling the operations of optical system 113

Optical emitter 125 can include laser elements such as a laser diode, solid-state laser, or other laser device, along with associated driving circuitry. Optical detector 126 can include light detection equipment, optical to electrical conversion circuitry, photon density wave characteristic detection equipment, and analog-to-digital conversion equipment. Optical detector 126 can include one or more photodiodes, phototransistors, avalanche photodiodes (APD), or other optoelectronic sensors, along with associated receiver circuitry such as amplifiers or filters. Optical couplers, cabling, or attachments can be included with optical emitter 125 and optical detector 126 to optically mate to associated ones of links 141-142.

Capacitance system 114 comprises one or more electrical interfaces for applying one or more electric field signals to tissue of a patient over any of electrical links 141-144. In some examples, capacitance system 114 drives one or more generally ring-shaped capacitor plates that are placed in proximity to tissue of a patient. Capacitance system 114 can include transceivers, amplifiers, modulators, capacitance monitoring systems and circuitry, impedance matching circuitry, human-interface circuitry, electrostatic discharge circuitry, and electromagnetic shield interface circuitry, including combinations thereof. Capacitance system 114 also can receive command and control information and instructions from processing system 111 over link 117 for controlling the operations of capacitance system 114.

Capacitance elements 121-122 each comprise electrically conductive ring elements which can be disposed about an optical emitter or optical detector to apply electric fields to tissue 130. Dielectric materials can be included around capacitance elements 121-122 to isolate capacitance elements 121-122 from tissue 130, from electrically conductive shield elements 123-124, or from optical emitter 125 and optical detector 126.

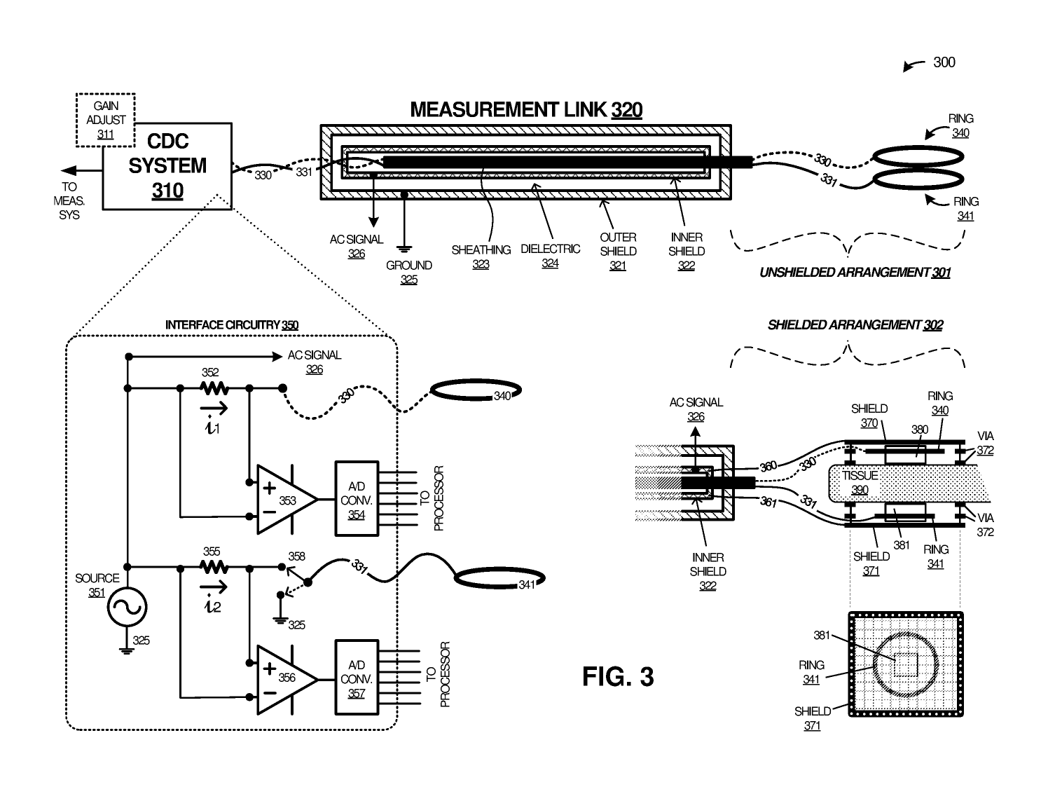

FIG. 3 is a system diagram illustrating physiological measurement system 300. System 300 includes capacitance-to-digital (CDC) system 310, measurement link 320, and two different sensor arrangements. Detailed examples of elements CDC system 310 are shown in interface circuitry 350 and gain adjust portion 311, each discussed below. Optical measurements can also be performed in conjunction with capacitive measurements by CDC system 310, but an optical system is omitted from FIG. 3 for clarity. Optical emitter 380 and optical detector 381 are shown as positioned proximate to capacitance sensing elements in FIG. 3, and are coupled to an associated optical measurement system, such as a pulse oximetry system or other measurement equipment.

A first capacitive sensor arrangement shown in FIG. 3 is unshielded arrangement 301 and a second capacitive sensor arrangement shown in FIG. 3 is shielded arrangement 302. Unshielded arrangement 301 is provided in FIG. 3 as an example of applying ring-shaped capacitance plates to tissue for measurement without electrical shielding from external electric fields. These external electric fields can arise from nearby objects or ambient conditions such as emissions from nearby equipment or lighting. To mitigate some interference from external electric fields and enhance capacitive coupling between rings 340-341, shielded arrangement 302 is also provided in FIG. 3 as an alternate example. Shielded arrangement 302 comprises a capacitive sensor arrangement with electrical shields 370-371 that shield ring elements 340-341. Although optical elements 380-381 are shown in shielded arrangement 302, these elements could also be included in unshielded arrangement 301.

During operation, tissue under measurement is placed in proximity to ring elements 340-341, such as tissue 390. In some examples, tissue 390 is placed between ring elements 340-341, while in other examples ring elements 340-341 are placed on the same side of tissue 390. As mentioned above, optical elements can be employed in conjunction with ring elements 340-341 to detect physiological signals for the tissue under measurement. Optical emitter 380 is configured to emit optical signals into tissue 390 and optical detector 381 is configured to detect the optical signals from tissue 390. As with ring elements 340-341, optical elements 380-381 can be placed on the same side of tissue 390, or be placed on opposing sides of tissue 390 such as pictured in FIG. 3.

A voltage potential can be established for tissue 390 to ensure a potential difference between tissue 390 and any of ring elements 340-341 during measurement. The voltage potential can be a reference potential, such as a signal ground. To establish a voltage potential for tissue 390, various techniques can be employed. In a first example, a conductive wrist strap can be placed onto the patient during measurement and the conductive wrist strap can be electrically connected to a reference potential voltage. In a second example, a further conductive plate lacking a dielectric layer between the further conductive plate and tissue 390 can be employed. The further conductive plate can be electrically coupled to a reference potential voltage. In a third example, the further conductive plate can include a dielectric layer between the further conductive plate and tissue 390 to capacitively couple tissue 390 to the reference potential voltage. One of ring elements 340-341 might comprise the further conductive plate when performing a single-ended capacitive measurement using only one of the ring elements.

CDC system 310 comprises a measurement system that employs electrical signals over measurement links 330-331 to identify physiological signals, and display equipment for presenting one or more physiological measurements to an operator. CDC system 310 can include elements discussed above for measurement system 110 of FIG. 1, although variations are possible. In one example, CDC system 310 includes gain adjust portion 311 which allows for application of a different gain to each of ring element 340-341. Ring elements 340-341 are shown as a similar size in FIG. 3, such as having the same diameter. An asymmetric gain is applied in CDC system 310, such as applying a hardware amplification factor to signals monitored by CDC system 310 or a software gain factor to data obtained by CDC system 310. Gain adjust portion 311 can be adjusted by an operator of system 300 to optimize signal properties associated with measurement of capacitance by ring elements 340-341. Alternatively, or in addition, the ring elements 340-341 may have different sizes or shapes to provide different gains.

Measurement link 320 is a link employed between CDC system 310 and sensor elements, such as ring elements 340-341 and optical elements 380-381, to carry measurement signals to and from CDC system 310. Measurement link 320 includes outer shield 321, inner shield 322, sheathing 323, and dielectric 324. Shields 321-322 comprise conductive shields, such as braid or foil that surround links 330-331. Links 330-331 form a twisted pair of conductors, with link 330 connected to ring element 340 and link 331 connected to ring element 341. Sheathing 323 and dielectric 324 comprise non-conductive materials which electrically isolate the conductive elements of measurement link 320 from each other and provide structural rigidity. In some examples, further signal links are included in measurement link for coupling optical elements 380-381 to associated measurement equipment.

In system 300, interface circuitry 350 is employed to drive measurement link 320 and ring elements 340-341 as well as to sense current as a measure of capacitance. At least three measurement configurations using ring elements 340-341 can be employed. In a first measurement configuration, ring element 340 and ring element 341 are both driven by source 351, and current draw which corresponds to capacitance is monitored for each capacitance element. In a second measurement configuration, ring element 340 is driven by source 351 but ring element 341 is coupled to a reference potential, namely ground 325. Current draw is monitored for ring element 340 which corresponds to a capacitance signal. Selectable node 358 can couple link 331 to either source 351 or ground 325. In a third measurement configuration, shields of measurement link 320 are also driven along with shield elements that accompany ring elements 340-341. As with the first measurement configuration, current draw is monitored for ring elements 340-341 which corresponds to capacitance signals.

Each of ring elements 340-341 can be driven with AC signal 326 from source 351 and associated current draws are monitored to identify motion noise in tissue of a patient using changes in capacitance for ring elements 340-341. Specifically, source 351 can drive AC signal 326 at a predetermined frequency through resistors 352 and 355 onto links 330-331 which drive ring elements 340-341. Resistors 352 and 355 comprise current sense resistors, which can be of a resistance value that provides a suitable voltage drop for detection by differential amplifiers 353 and 356 based on currents i₁ and i₂. When driven by source 351, ring elements 340-341 emit an associated electrostatic field based on the driven AC signal. During application of the electrostatic field into tissue, such as tissue 390, current draw across the associated resistor 352 and 355 is monitored using differential amplifiers 353 and 356 and provided to analog-to-digital converters (A/D cony.) 354 and 357. A/D converters 354 and 357 convert the associated differential current draws into a digital format for delivery to a processor or processing circuitry, such as that found in CDC system 310 or other processing elements.

When shielded arrangement 302 is employed, AC signal 326 is also applied to inner shield 322 of measurement link 320, while outer shield 321 is coupled to ground 325. Furthermore, inner shield 322 is electrically coupled to shields 370-371 by associated ones of links 360-361. Shield 370 is positioned near ring element 340 to shield ring element 340 from external electric fields and from field lines associated with ring element 340 from coupling to external objects, such as medical personnel, medical equipment, and other external object. Likewise, shield 371 is positioned near ring element 341 to shield ring element 341. The energy of ring elements 340-341 is directed into tissue 390. Shields 370-371 also incorporates side shield elements that also shield a left/right side of ring element 340-341.

In this example, shields 370-371 are each formed from a first piece of conductive material that forms the main shield portion, and further materials that form the sides. For example, a conductive plane on a printed circuit board could comprise the main portion of shield 371, as shown in the separate view in FIG. 3. Vias 372 can link a first layer of the printed circuit board to subsequent layers which have associated conductive planes that allow for an aperture that can house an associated ring element 341 and optical element 381. Vias include electrically conductive through-holes which electrically couple layers of printed circuit boards or other layered circuitry. Shield 370 can comprise similar elements as shown in the separate view for shield 371. Instead of a printed circuit board, other conductive elements can be employed for shields 370-371, such as conductive foil, flex circuits, mesh conductors, among others, including combinations thereof. The size of overlap of shields 370-371 over ring elements 340-341 is approximately 20% in FIG. 3, although other overlap amounts are possible.

When shields 370-371 are energized with a similar signal as ring elements 340-341, the electrical potential difference between these elements is also minimized. Specifically, shields 370-371 are driven actively with AC signal 326 as well as ring elements 340-341. This active driving of both shield and capacitance plate allows for enhanced measurement of tissue 390 while minimizing interference from external objects.

As mentioned above, a current draw for each of the capacitive ring elements 340-341 is monitored to determine an associated capacitance signals of ring elements 340-341 which indicates at least motion noise of tissue 390. The capacitance signals can vary based on different changes related to tissue 390 or the environment of tissue 390. While shields 370-371 minimize changes in the capacitance signals from external objects and external electric fields, ring elements 340-341 detect changes in tissue 390 that are related to motion of tissue 390 as well as other physiological changes of tissue 390, such as volume changes of tissue 390 during pulsatile activity of the patient, bending of tissue 390 due to movement by the patient, or other motion of the patient. Additional sources of noise are found in the capacitance signals of ring elements 340-341, but are typically of a lesser magnitude than motion of tissue 390.

A time-varying capacitance signal for each of ring elements 340-341 can be used to reduce noise in a time-varying optical measurement of tissue 390, such as in a PPG measured for tissue 390 using optical elements 380-381. The PPG measured for tissue 390 can include various noise components, such as caused by motion of tissue 390. However, the PPG typically also includes other signal components, namely signal components that indicate a pulse of the patient, a breathing rate of the patient, and other signal components. Capacitance signals measured by ring elements 340-341 are used to reduce the magnitude of the motion components in the PPG.

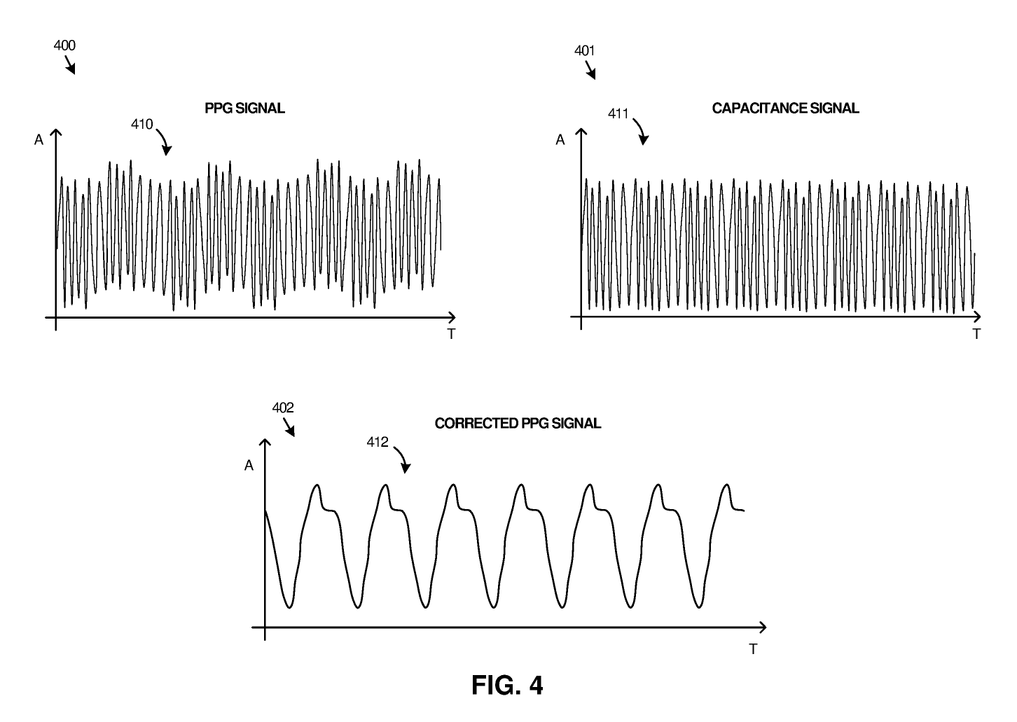

In a first example, FIG. 4 is presented. FIG. 4 shows a measured PPG signal 410 in graph 400, such as measured by optical elements 380-381 and an associated PPG measurement system. It should be noted that the various axes among graphs of FIG. 4 might not be to scale. PPG signal 410 is noisy as it includes various motion-based noise components of varying amplitudes (A) as plotted over time (T) in graph 400. Other noise components are typically found in the PPG signal, but motion-based noise is discussed in this example. Capacitance signal 411 is shown in graph 401 which also includes motion-based noise components. Since capacitance signal 411 is measured concurrently and from the same tissue as PPG signal 410, much of the motion-based noise components are correlated in both time and frequency among PPG signal 410 and capacitance signal 411. Corrected PPG 412 is then determined and shown in graph 402. Corrected PPG 412 can be determined by subtracting a scaled version of capacitance signal 411 from PPG signal 410. Corrected PPG 412 can be determined by subtracting various frequency components of capacitance signal 411 from frequency components of PPG signal 410. Other correction methods can be employed to derive corrected PPG 412 from PPG signal 410 using capacitance signal 411 as a correction factor or noise-reduction signal, such as filtering, adaptive filtering, spectral subtraction, or other methods, including combinations thereof.

It should be noted that capacitance signal 411 comprises a differential signal formed from capacitance signals monitored for both ring elements 340-341. This differential signal is determined by monitoring a current draw for each of ring elements 340-341 while energized using ac signal 326. The current draws for ring elements 340-341 are compared to identify a difference signal which represents a differential capacitance signal among ring elements 340-341. This difference signal can be determined in hardware, such as in further elements included in interface circuitry 350, or can be determined in software once the associated signals are digitized by A/D converters 354 and 357. An asymmetric gain is also applied to each capacitance signal monitored for ring elements 340-341 to establish the difference signal. In FIG. 1, the asymmetric gain is provided by at least different diameters for capacitance rings. However, in FIG. 3, similar diameter capacitance rings are employed and a different gain is applied in either interface circuitry 350 (such as in differential amplifiers 353 and 356) or in software of an associated processor or processing system.

The asymmetric gain can comprise a first gain applied to signals measured for ring element 340 and a second gain applied to signals measured for ring element 341. The first gain and the second gain can be established to maximize signal quality for the signals under measurement. For example, the gains can be established to maximize resolution of motion noise associated with tissue 390 being moved by the patient while minimizing resolution of motion noise due to the cardiac pulse within tissue 390. The gains can be selected based on desired frequency sensitivity of these various types of motion noise, such as to minimize sensitivity to frequency ranges associated with pulse motion within tissue 390, while maximizing sensitivity to frequency ranges associated with movement of tissue 390 within the environment. Empirical measurements can be performed on the patient or prior to measurement of tissue 390 which can establish desired gains or to calibrate the gains applied to capacitance signals on a per-patient basis. Similarly, a sizing of ring elements 340-341 can be established based on a desired gain, differential gain, or upon sensitivity to certain frequency components associated with motion noise.

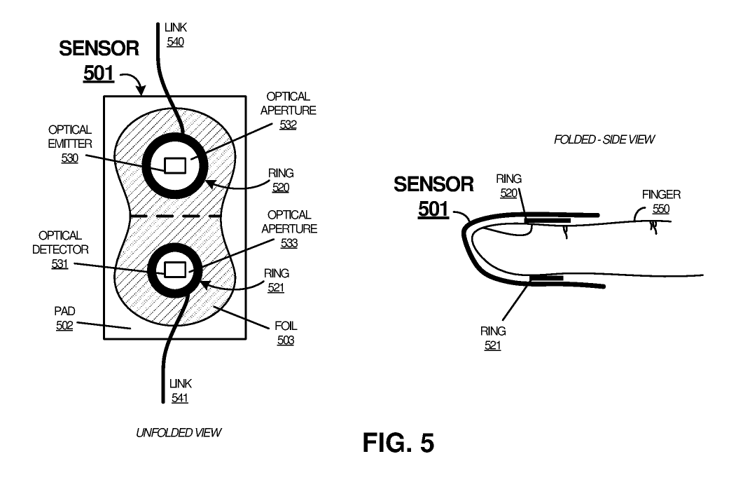

FIG. 5 is a system diagram illustrating physiological sensor 501. Sensor 501 is used to measure physiological signals from a patient, such as measured by the various optical sensors and capacitance sensors described herein. Sensor 501 is generally flexible and can be applied to tissue of a patient, such as finger 550 shown in FIG. 5. Sensor 501 can be applied to other tissue portions of a patient, such as a forehead, ear, limb, chest, or other location. Sensor 501 can be applied to tissue in a generally folded configuration, as shown around finger 550 in FIG. 5, or in a flat configuration, such as to a forehead.

Sensor 501 includes pad 502, foil 503, capacitive rings 520-521, optical emitter 530, optical detector 531, and links 540-541. Pad 502 comprises a material for coupling sensor 501 to tissue of a patient and a structural member for holding the remaining elements of sensor 501. Pad 502 can comprise an adhesive pad which is stuck onto tissue of a patient, or can comprise a non-adhesive pad which is held onto tissue with other equipment not shown in FIG. 5, such as clamps, bands, springs, or other elements. Foil 503 comprises a bendable metallic element which also electrically shields at least optical detector 531 from external electromagnetic interference. Foil 503 can be configured to shield any of the components of sensor 501, and separates pad 502 from the sensing elements. Dielectric materials can also be included in sensor 501 which separate the various sensing elements from foil 503.

Optical emitter 530 is positioned within optical aperture 532 to allow for emission of optical energy into tissue. Optical detector 531 is positioned within optical aperture 533 for detection of optical energy from tissue. Optical apertures 532-533 can comprise optically transmissive portions of sensor 501 to allow for the optical elements to optically interface with tissue, and can include lenses, prisms, transparent films, and the like. In some examples, optical apertures 532-533 can include metallic mesh portions which electrically shield the optical elements by creating a Faraday cage for the optical elements between foil 503 and the associated metallic mesh portion.

Capacitive rings 520-521 each comprise a ring-shaped thin metallic sheet, metallic plate, or metallic grid, along with other configurations, which is separated from foil 503 by a non-conductive material. In FIG. 5, each of capacitive rings 520-521 is of a different diameter, with ring 520 which surrounds optical emitter 530 being of a larger diameter than ring 521 which surrounds optical detector 531. It should be understood that other diameters are possible. When positioned onto tissue, each of capacitive rings 520-521 is configured to emit an electric field into the space proximate to capacitive rings 520-521, which generally includes the tissue near capacitive rings 520-521. Further non-conductive material can be employed to separate capacitive rings 520-521 from making conductive contact with tissue, such as a dielectric material that coats each of capacitive rings 520-521.

Link 540 is coupled to capacitive ring 520 and link 541 is coupled to capacitive ring 521. Links 540-541 can be employed to drive measurement signals to capacitive rings 520-521, and links 540-541 are monitored by a measurement system to identify capacitance signals associated with capacitive rings 520-521. In other examples, ones of links 540-541 can be coupled to reference potentials, such as an electrical ground. Links for optical emitter 530 and optical detector 531 are omitted from FIG. 5 for clarity, but could be included in link 540 or link 541. Moreover, link 540-541 may be combined into a single composite link along with links for optical emitter 530 and optical detector 531.

The side view of FIG. 5 shows sensor 501 applied to finger 550 to emphasize the placement of capacitive rings 520-521 proximate to finger 550. Capacitive rings 520-521 can be employed in a differential capacitive arrangement, where each of capacitive rings 520-521 is driven with a measurement signal. Capacitive rings 520-521 can be employed in a single-ended capacitive arrangement, where a first of capacitive rings 520-521 is driven by a measurement signal and a second of capacitive rings 520-521 is coupled to an electrical ground. Other configurations of capacitive rings 520-521 can be employed, and any one of capacitive rings may be omitted.

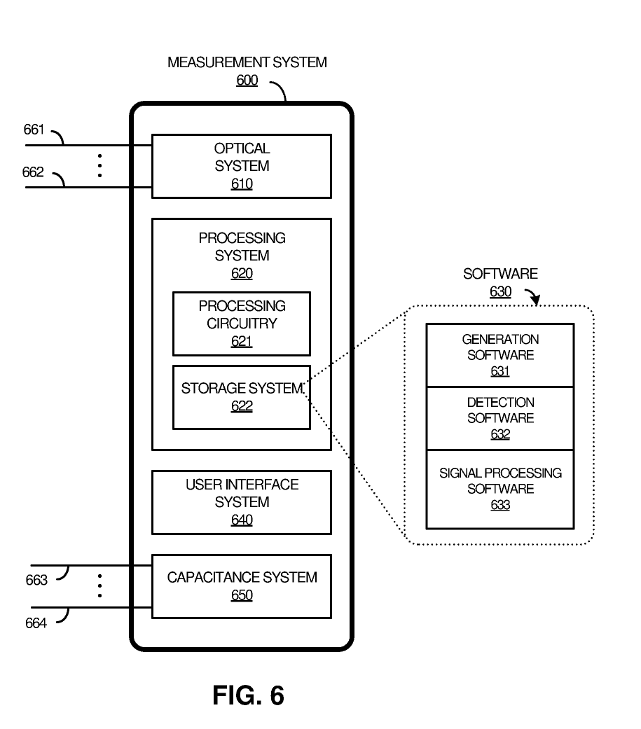

FIG. 6 is a block diagram illustrating measurement system 600, as an example of elements of measurement system 110 in FIG. 1 or CDC system 310 in FIG. 3, although these can use other configurations. Measurement system 600 includes optical system 610, processing system 620, software 630, user interface 640, and capacitance system 650. Processing system 620 further includes processing circuitry 621 and storage system 622. In operation, processing circuitry 621 is operatively linked to optical system 610, user interface 640, and capacitance system 650 by one or more communication interfaces, which can comprise a bus, discrete connections, network links, software interfaces, or other circuitry. Measurement system 600 can be distributed or consolidated among equipment or circuitry that together forms the elements of measurement system 600. Measurement system 600 can optionally include additional devices, features, or functionality not discussed here for purposes of brevity.

Optical system 610 comprises a communication interface for communicating with other circuitry and equipment, such as with optical system 113 of FIG. 1. Optical system 610 can include transceiver equipment exchanging communications over one or more of the associated links 661-662. It should be understood that optical system 610 can include multiple interfaces, pins, transceivers, or other elements for communicating with multiple external devices. Optical system 610 also receives command and control information and instructions from processing system 620 or user interface 640 for controlling the operations of optical system 610. Links 661-662 can each use various protocols or communication formats as described herein for link 116 or links 145-146 of FIG. 1, including combinations, variations, or improvements thereof. In some examples, optical system 610 includes optical interface equipment, such as that discussed above for optical system 113.

Processing system 620 includes processing circuitry 621 and storage system 622. Processing circuitry 621 retrieves and executes software 630 from storage system 622. In some examples, processing circuitry 621 is located within the same equipment in which optical system 610, user interface 640, or capacitance system 650 are located. In further examples, processing circuitry 621 comprises specialized circuitry, and software 630 or storage system 622 can be included in the specialized circuitry to operate processing circuitry 621 as described herein. Storage system 622 can include a non-transitory computer-readable medium such as a disk, tape, integrated circuit, server, flash memory, or some other memory device, and also may be distributed among multiple memory devices.

Software 630 may include an operating system, logs, utilities, drivers, networking software, tables, databases, data structures, and other software typically loaded onto a computer system. Software 630 can contain application programs, server software, firmware, processing algorithms, or some other form of computer-readable processing instructions. When executed by processing circuitry 621, software 630 directs processing circuitry 621 to operate as described herein, such as instruct optical or capacitance systems to generate optical or electrical signals for measurement of physiological parameters of patients, receive signals representative of optical or capacitance measurements of patients, and process at least the received signals to determine physiological parameters of patients, among other operations.

In this example, software 630 includes generation module 631, detection module 632, and signal processing module 633. It should be understood that a different configuration can be employed, and individual modules of software 630 can be included in different equipment in measurement system 600. Generation module 631 determines parameters for optical or capacitance signals, such as modulation parameters, signal strengths, amplitude parameters, voltage parameters, on/off conditions, or other parameters used in controlling the operation of optical systems and capacitance systems over ones of links 661-664. Generation module 631 directs optical system 610 and capacitance system 650 to perform physiological measurements, and can selectively drive various detection sensors, emitters, capacitors, and other sensor elements. Detection module 632 receives data or signals representing optical and capacitive measurements. Signal processing module 633 processes the received characteristics of optical and capacitance signals to determine physiological parameters, filter optical data based on capacitance data, and reduce motion noise in optical measurements using capacitance measurements, among other operations.

User interface 640 includes equipment and circuitry to communicate information to a user of measurement system 600, such as alerts, measurement results, and measurement status. Examples of the equipment to communicate information to the user can include displays, indicator lights, lamps, light-emitting diodes, haptic feedback devices, audible signal transducers, speakers, buzzers, alarms, vibration devices, or other indicator equipment, including combinations thereof. The information can include blood parameter information, waveforms, summarized blood parameter information, graphs, charts, processing status, or other information. User interface 640 also includes equipment and circuitry for receiving user input and control, such as for beginning, halting, or changing a measurement process or a calibration process. Examples of the equipment and circuitry for receiving user input and control include push buttons, touch screens, selection knobs, dials, switches, actuators, keys, keyboards, pointer devices, microphones, transducers, potentiometers, non-contact sensing circuitry, or other human-interface equipment.

Capacitance system 650 comprises a communication interface for communicating with other circuitry and equipment, such as with capacitance system 114 of FIG. 1. Capacitance system 650 can include transceiver equipment exchanging communications over one or more of the associated links 663-664. It should be understood that capacitance system 650 can include multiple interfaces, pins, transceivers, or other elements for communicating with multiple external devices. Capacitance system 650 also receives command and control information and instructions from processing system 650 or user interface 640 for controlling the operations of capacitance system 650. Links 663-664 can each use various protocols or communication formats as described herein for link 117 or links 141-144 of FIG. 1, including combinations, variations, or improvements thereof. In some examples, capacitance system 610 includes capacitance interface equipment, such as that discussed above for capacitance system 114.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above.

What is claimed is:

1. A physiological sensor configured to be positioned onto tissue of a patient, the sensor comprising:
a first conductive element with an associated first gain property and disposed about an optical emitter;
a second conductive element with an associated second gain property and disposed about an optical detector; and
a sensor body coupled to the first conductive element and the second conductive element and configured to interface with tissue of the patient.

2. The sensor of claim 1, wherein the first gain property comprises a conductive area of the first conductive element, and wherein the second gain property comprises a conductive area of the second conductive element, the conductive area of the second conductive element being less than the conductive area of the first conductive element.

3. The sensor of claim 1, wherein the first conductive element and the second conductive element comprise a same conductive area, wherein the first gain property comprises a gain factor applied to signals detected with the first conductive element, and wherein the second gain property comprises a gain factor applied to signals detected with the second conductive element.

4. The sensor of claim 1, comprising:
at least one of the first conductive element and the second conductive element configured to emit an electric field into the tissue of the patient when driven by an electric signal from a measurement system.

5. The sensor of claim 1, further comprising:
a first conductive shield element associated with the first conductive element and configured to direct an electric field emitted by the first conductive into the tissue of the patient.

6. The sensor of claim 5, further comprising:
a second conductive shield element associated with the second conductive element and configured to shield the second conductive element from electric fields other than the electric field emitted by the first conductive element.

7. The sensor of claim 1, wherein the first conductive element is configured as a first capacitor plate of a differential capacitive arrangement, wherein the second conductive element is configured as a second capacitor plate of the differential capacitive arrangement.

8. A measurement system, comprising:
a capacitance system configured to measure a first capacitance signal from a first capacitance element positioned proximate to an optical emitter that emits an optical signal into tissue of a patient;
the capacitance system configured to measure a second capacitance signal from a second capacitance element positioned proximate to an optical detector that detects the optical signal after propagation through the tissue of the patient, the second capacitance element having an associated gain asymmetric to that of the first capacitance element;
a processing system configured to compare the first capacitance signal to the second capacitance signal to identify a differential capacitance signal;
the processing system configured to use at least the differential capacitance signal to identify a corrected PPG by reducing a magnitude of a noise component in a photoplethysmogram (PPG) that is derived from the optical signal.

9. The measurement system of claim 8, comprising:
the capacitance system configured to drive the first capacitance element with an alternating current (AC) signal to establish a first electric field in the tissue of the patient, wherein the first capacitance signal is derived from a first current draw detected in the AC signal driving the first capacitance element;
the capacitance system configured to drive the second capacitance element with the AC signal to establish a second electric field in the tissue of the patient, wherein the second capacitance signal is derived from a second current draw detected in the AC signal driving the second capacitance element.

10. The measurement system of claim 9, comprising:
the capacitance system configured to drive a first conductive shield with the AC signal, the first conductive shield positioned on a side of the first capacitance element opposite of the tissue and separated from the first capacitance element by dielectric material;
the capacitance system configured to drive a second conductive shield with the AC signal, the second conductive shield positioned on a side of the second capacitance element opposite of the tissue and separated from the second capacitance element by further dielectric material.

11. The measurement system of claim 10, wherein the first conductive shield comprises a first conductive ring larger than the first capacitance element, and wherein the second conductive shield comprises a second conductive ring larger than the second capacitance element.

12. The measurement system of claim 10, wherein the first conductive shield comprises a first conductive plate larger than the first capacitance element and at least partially enshrouding an outer edge of the first capacitance element, and wherein the second conductive shield comprises a second conductive plate larger than the second capacitance element and at least partially enshrouding an outer edge of the second capacitance element.

13. The measurement system of claim 8, wherein the associated gain of the second capacitance element asymmetric to that of the first capacitance element is established based at least on the first capacitance having a different conductive area than the second capacitance element.

14. The measurement system of claim 8, wherein the associated gain of the second capacitance element asymmetric to that of the first capacitance element is established based at least on signals detected for the first capacitance having a different associated amplification factor in the capacitance system than signals detected for the second capacitance element.

15. The measurement system of claim 8, wherein the associated gain of the second capacitance element asymmetric to that of the first capacitance element is established based at least on signals processed for the first capacitance having a different associated gain factor than signals processed for the second capacitance element, the different associated gain factor applied in software executed on the processing system.

16. The measurement system of claim 8, comprising:
the processing system configured to report the corrected PPG for display to a user of the measurement system.

17. A physiological measurement apparatus, comprising:
a generally ring-shaped first capacitor plate configured to interface with tissue of a patient to emit an electric field proximate to the tissue of the patient, the first capacitor plate having a first associated gain property;
a generally ring-shaped second capacitor plate configured to interface with the tissue of the patient and having a second associated gain property different than the first gain property;
a measurement system electrically coupled to the first capacitor plate and the second capacitor plate and configured to:
generate an electric signal referenced to a ground potential;
drive the electric signal to the first capacitor plate for emission as the electric field;
electrically couple the second capacitor plate to the ground potential;
monitor properties of the electric signal during emission into the tissue of the patient to identify a capacitance signal associated with the first capacitor plate; and
process the capacitance signal to determine one or more physiological metrics associated with the patient.

18. The apparatus of claim 17, comprising:
the measurement system configured to monitor at least a current draw of the electric signal during emission by the first capacitor plate to derive the capacitance signal.

19. The apparatus of claim 17, wherein the one or more physiological metrics comprise a pulse rate, a breathing rate, a capacitive plethysmogram (CPG), and motion of the tissue of the patient.

20. The apparatus of claim 17, comprising:
correlating the one or more physiological metrics with photoplethysmogram (PPG) data of the tissue of the patient to reduce noise in the PPG data.
